(19) United States
(12) Patent Application Publication
(10) Pub. No.: US 2024/0281973 A1
(43) Pub. Date: Aug. 22, 2024

(54) INFORMATION PROCESSING PROGRAM, INFORMATION PROCESSING DEVICE, INFORMATION PROCESSING METHOD, AND MICROSCOPE SYSTEM

(71) Applicant: SONY GROUP CORPORATION, TOKYO (JP)

(72) Inventors: RYOTA YAMADA, TOKYO (JP); JUNICHIRO ENOKI, TOKYO (JP)

(21) Appl. No.: 18/566,254

(22) PCT Filed: Mar. 2, 2022
(86) PCT No.: PCT/JP2022/008730

§ 371 Date: Dec. 1, 2023

(30) Foreign Application Priority Data
Jun. 9, 2021 (JP) 2021-096758

(51) Int. Cl. G06T 7/00; G02B 21/36

(52) U.S. Cl. CPC G06T 7/0014; G02B 21/365; G06T 2207/10056; G06T 2207/20012; G06T 2207/20021; G06T 2207/20104; G06T 2207/30024

(57) ABSTRACT

There are provided an information processing program, an information processing device, an information processing method, and a microscope system capable of more efficiently performing parameter adjustment. The information processing program is an information processing program for causing a computer to execute an acquisition step of acquiring a first image obtained by imaging a specimen tissue, an analysis processing step of performing analysis processing on the first image using a predetermined parameter, and a determination step of determining whether to allow the analysis processing to be performed with the predetermined parameter on the basis of first characteristic information of a tissue form present in the first image and second characteristic information of a tissue form corresponding to the predetermined parameter.

### TECHNICAL FIELD

The present disclosure relates to an information processing program, an information processing device, an information processing method, and a microscope system.

### BACKGROUND ART

There is known a microscope system that images an observation target placed on a glass slide with a microscope and generates a whole slide imaging (WSI) image that is a digitized pathological image. This microscope system can extract information by performing various image analysis. Furthermore, in order to perform various types of image analysis on this microscope system, it is necessary for the user to adjust parameters.

### CITATION LIST

### Patent Document

Patent Document 1: Japanese Patent Application Laid-Open No. 2013-007849

### SUMMARY OF THE INVENTION

### Problems to be Solved by the Invention

However, if parameter adjustment is performed using all tissue regions on the WSI image, processing time is required. Accordingly, parameters are adjusted at a higher speed by using a minute region of interest (ROI) on the WSI image. Adjustment of this parameter may increase a processing load of the observer.

Therefore, the present disclosure provides an information processing program, an information processing device, an information processing method, and a microscope system capable of more efficiently performing parameter adjustment.

### Solutions to Problems

In order to solve the above problem, according to the present disclosure, there is provided an information processing program for causing a computer to execute:
an acquisition step of acquiring a first image obtained by imaging a specimen tissue;
an analysis processing step of performing analysis processing on the first image using a predetermined parameter; and
a determination step of determining whether to allow the analysis processing to be performed with the predetermined parameter on the basis of first characteristic information of a tissue form present in the first image and second characteristic information of a tissue form corresponding to the predetermined parameter.

The determination step may determine not to allow the analysis processing to be performed with the predetermined parameter in a case where the first characteristic information and the second characteristic information are not within a predetermined distance on a statistical distribution.

A display control step of causing a display unit to display a display form in which setting of a new parameter for the analysis processing step is recommended in a case where the determination step determines not to allow the analysis processing to be performed with the predetermined parameter may be further included.

The first characteristic information may be generated on the basis of an image in a predetermined region of the first image, and

at least one of an image in the predetermined region or an image indicating the predetermined region may be displayed as the display form.

A language related to recommending setting of a parameter may be displayed as the display form.

The display control step may cause the display unit to display an image indicating position information of the first characteristic information in the statistical distribution and the display form side by side.

The first characteristic information may be a plurality of pieces of characteristic information selected from a plurality of pieces of characteristic information based on a plurality of processing regions in the first image, and

the display control step may cause the display unit to display images in a processing region corresponding to the selected plurality of pieces of characteristic information side by side.

The first characteristic information may be a plurality of pieces of characteristic information selected from each of clustered regions by clustering a plurality of pieces of characteristic information based on each of a plurality of processing regions in the first image into a plurality of regions, and

the display control step may display the processing regions of each of the clustered regions on the first image in association with each other.

The first characteristic information may be a plurality of pieces of characteristic information selected from a plurality of pieces of characteristic information based on a plurality of processing regions in the first image, and

the determination step may change a determination reference in such a manner that the first characteristic information is not selected in a case where it is determined not to allow the analysis processing to be performed with the predetermined parameter and a parameter based on a processing region corresponding to the first characteristic information is not used for the analysis processing.

The first characteristic information may be a plurality of pieces of characteristic information selected from a plurality of pieces of characteristic information based on a plurality of processing regions in the first image, and

the determination step changes an imaging condition of an imaging device that has captured the first image in such a manner that the first characteristic information is not selected in a case where it is determined not to allow the analysis processing to be performed with the predetermined parameter and a parameter based on a processing region corresponding to the first characteristic information is not used for the analysis processing.

The second characteristic information may be a plurality of pieces of characteristic information calculated from different captured images.

The determination step may change the predetermined distance according to a number of pieces of the second characteristic information corresponding to the predetermined parameter.

A storage step of storing a plurality of parameters used in the analysis processing step in a storage unit in association with the second characteristic information corresponding to the parameters may be further included, in which

in a case where the determination step determines not to allow the analysis processing to be performed with the predetermined parameter, the determination step may cause the storage unit to store a new parameter of the analysis processing step corresponding to the first characteristic information in association with the first characteristic information.

The determination step may determine whether characteristic information similar to the first characteristic information is stored in the storage unit in a case where it is determined not to allow the analysis processing to be performed with the predetermined parameter, and in a case where the characteristic information is not stored, the determination step may cause the storage unit to store a new parameter of the analysis processing step corresponding to the first characteristic information.

In a case where it is determined that the characteristic information similar to the first characteristic information is stored in the storage unit, the determination step may allow the analysis processing to be performed on the basis of a parameter corresponding to the similar characteristic information.

The determination step may allow the analysis processing to be performed with the predetermined parameter in a case where the first characteristic information and the second characteristic information are within a predetermined distance on a statistical distribution.

A display control step of causing a display unit to display a display form indicating that it is possible to perform the analysis processing with the predetermined parameter in a case where the determination step determines to allow the analysis processing to be performed with the predetermined parameter may be further included.

The first characteristic information and the second characteristic information may be at least one of a luminance value, cell density, cell circularity, a cell circumferential length, or a local feature amount, and the distance may be at least one of a Mahalanobis distance or a Euclidean distance.

In order to solve the above problem, according to the present disclosure, there is provided an information processing device including:
an acquisition unit that acquires a first image obtained by imaging a specimen tissue;
an analysis processing unit that performs analysis processing on the first image using a predetermined parameter; and
a determination unit that determines whether to allow the analysis processing to be performed with the predetermined parameter on the basis of first characteristic information of a tissue form present in the first image and second characteristic information of a tissue form corresponding to the predetermined parameter.

In order to solve the above problems, according to the present disclosure, there is provided a microscope system including:
a microscope device that acquires a first image obtained by imaging a specimen tissue; and
an information processing device, in which
the information processing device includes
an acquisition unit that acquires a first image obtained by imaging a specimen tissue,
an analysis processing unit that performs analysis processing on the first image using a predetermined parameter, and
a determination unit that determines whether to allow the analysis processing to be performed with the predetermined parameter on the basis of first characteristic information of a tissue form present in the first image and second characteristic information of a tissue form corresponding to the predetermined parameter.

### BRIEF DESCRIPTION OF DRAWINGS

FIG. 1 is a diagram schematically showing the overall configuration of a microscope system.

FIG. 2 is a diagram showing an example of an imaging method.

FIG. 3 is a diagram showing an example of an imaging method.

FIG. 4 is a block diagram illustrating a more detailed configuration example of an information processing unit.

FIG. 5 is a diagram illustrating an example of a plurality of ROIs for calculating a feature amount.

FIG. 6 is a diagram illustrating an example of a user interface for selecting characteristic information for selecting a feature amount.

FIG. 7 is a diagram schematically illustrating a distribution of feature amounts of each ROI calculated by a feature amount calculation unit.

FIG. 8 is a diagram schematically illustrating positions in a feature amount distribution in a representative ROI.

FIG. 9 is a diagram schematically illustrating a position in a feature amount distribution corresponding to another parameter in a representative ROI.

FIG. 10 is a flowchart illustrating a processing example of an information processing device according to a first embodiment.

FIG. 11 is a block diagram illustrating a configuration example of an information processing unit according to a second embodiment.

FIG. 12 is a diagram illustrating an example of a screen region.

FIG. 13 is a diagram illustrating an example in which specific information is displayed in each region illustrated in FIG. 12.

FIG. 14 is a flowchart illustrating a processing example of an information processing device according to the second embodiment.

FIG. 15 is a flowchart illustrating a processing example of the information processing device in a case where a recommended ROI is not employed.

FIG. 16 is a block diagram illustrating a configuration example of an information processing device 5120 according to a third embodiment.

FIG. 17 is a flowchart illustrating a relearning example of the information processing device 5120 in a case where a recommended ROI is not employed.

FIG. 18 is a flowchart illustrating an example of determining whether there is similarity information in the characteristic information stored in the storage unit.

FIG. 19 is a diagram illustrating an example of a determination distance used by a similarity determination processing unit.

### MODE FOR CARRYING OUT THE INVENTION

Hereinafter, embodiments of an information processing program, an information processing device, an information processing method, and a microscope system will be described with reference to the drawings. Hereinafter, main components of the information processing program, the information processing device, the information processing method, and the microscope system will be mainly described, but the information processing program, the information processing device, the information processing method, and the microscope system may include components and functions that are not illustrated or described. The following description does not exclude components and functions that are not illustrated or described.

### First embodiment

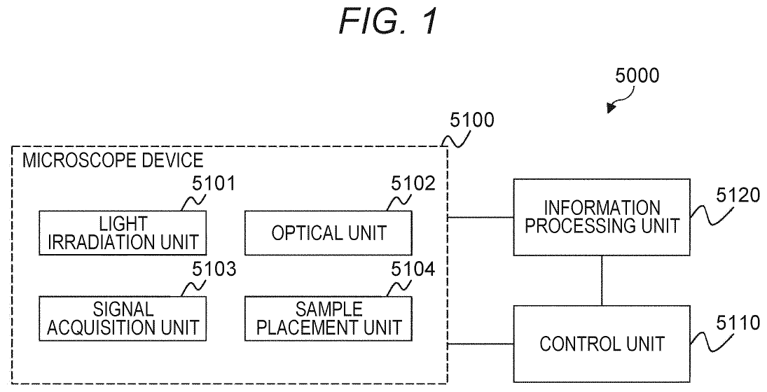

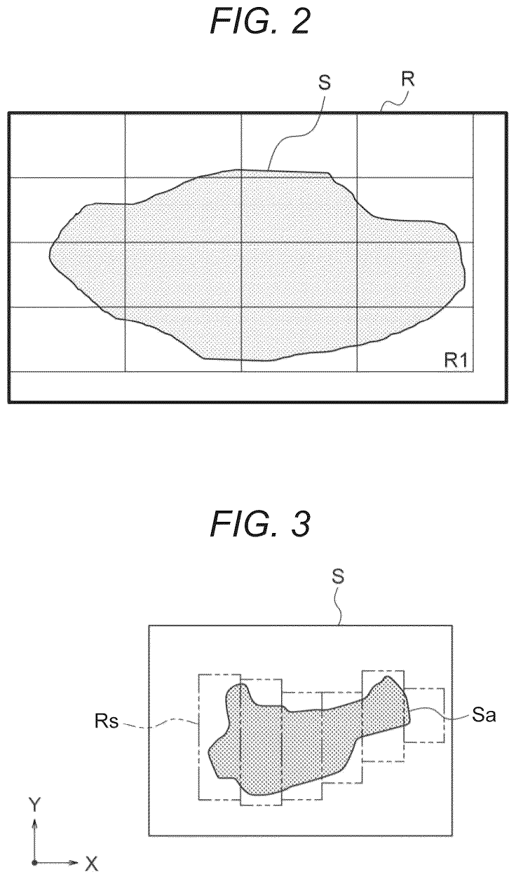

FIG. 1 shows an example configuration of a microscope system of the present disclosure. A microscope system 5000 shown in FIG. 1 includes a microscope device 5100, a control unit 5110, and an information processing unit 5120. The microscope device 5100 includes a light irradiation unit 5101, an optical unit 5102, and a signal acquisition unit 5103. The microscope device 5100 may further include a sample placement unit 5104 on which a biological sample S is placed. Note that the configuration of the microscope device is not limited to that shown in FIG. 1. For example, the light irradiation unit 5101 may exist outside the microscope device 5100, and a light source not included in the microscope device 5100 may be used as the light irradiation unit 5101. Alternatively, the light irradiation unit 5101 may be disposed so that the sample placement unit 5104 is sandwiched between the light irradiation unit 5101 and the optical unit 5102, and may be disposed on the side at which the optical unit 5102 exists, for example. The microscope device 5100 may be configured by one or two or more of bright field observation, phase difference observation, differential interference observation, polarization observation, fluorescence observation, and dark field observation.

The microscope system 5000 may be configured as a so-called whole slide imaging (WSI) system or a digital pathology system, and may be used for pathological diagnosis. Alternatively, the microscope system 5000 may be designed as a fluorescence imaging system, or particularly, as a multiple fluorescence imaging system.

For example, the microscope system 5000 may be used to make an intraoperative pathological diagnosis or a telepathological diagnosis. In the intraoperative pathological diagnosis, the microscope device 5100 can acquire the data of the biological sample S acquired from the subject of the operation while the operation is being performed, and then transmit the data to the information processing unit 5120. In the telepathological diagnosis, the microscope device 5100 can transmit the acquired data of the biological sample S to the information processing unit 5120 located in a place away from the microscope device 5100 (such as in another room or building). In these diagnoses, the information processing unit 5120 then receives and outputs the data. On the basis of the output data, the user of the information processing unit 5120 can make a pathological diagnosis.

### Biological Sample

The biological sample S may be a sample containing a biological component. The biological component may be a tissue, a cell, a liquid component of the living body (blood, urine, or the like), a culture, or a living cell (a myocardial cell, a nerve cell, a fertilized egg, or the like).

The biological sample may be a solid, or may be a specimen fixed with a fixing reagent such as paraffin or a solid formed by freezing. The biological sample can be a section of the solid. A specific example of the biological sample may be a section of a biopsy sample.

The biological sample may be one that has been subjected to a treatment such as staining or labeling. The treatment may be staining for indicating the morphology of the biological component or for indicating the substance (surface antigen or the like) contained in the biological component, and can be hematoxylin-eosin (HE) staining or immunohistochemistry staining, for example. The biological sample may be one that has been subjected to the above treatment with one or more reagents, and the reagent (s) can be a fluorescent dye, a coloring reagent, a fluorescent protein, or a fluorescence-labeled antibody.

The specimen may be prepared from a specimen or a tissue sample collected from a human body for the purpose of pathological diagnosis, clinical examination, or the like. Alternatively, the specimen is not necessarily of the human body, and may be derived from an animal, a plant, or some other material. The specimen may differ in property, depending on the type of the tissue being used (such as an organ or a cell, for example), the type of the disease being examined, the attributes of the subject (such as age, gender, blood type, and race, for example), or the subject's daily habits (such as an eating habit, an exercise habit, and a smoking habit, for example). The specimen may be managed by being affixed with identification information (bar-code information, QR-code (trademark) information, or the like) by which each specimen can be identified.

### Light Irradiation Unit

The light irradiation unit 5101 is a light source for illuminating the biological sample S, and is an optical unit that guides light emitted from the light source to a specimen. The light source can illuminate a biological sample with visible light, ultraviolet light, infrared light, or a combination thereof. The light source may be one or more of the following: a halogen lamp, a laser light source, an LED lamp, a mercury lamp, and a xenon lamp. The light source in fluorescent observation may be of a plurality of types and/or wavelengths, and the types and the wavelengths may be appropriately selected by a person skilled in the art. The light irradiation unit may have a configuration of a transmissive type, a reflective type, or an epi-illumination type (a coaxial epi-illumination type or a side-illumination type).

### Optical Unit

The optical unit 5102 is designed to guide the light from the biological sample S to the signal acquisition unit 5103. The optical unit may be designed to enable the microscope device 5100 to observe or capture an image of the biological sample S.

The optical unit 5102 may include an objective lens. The type of the objective lens may be appropriately selected by a person skilled in the art, in accordance with the observation method. The optical unit may also include a relay lens for relaying an image magnified by the objective lens to the signal acquisition unit. The optical unit may further include optical components other than the objective lens and the relay lens, and the optical components may be an eyepiece, a phase plate, a condenser lens, and the like.

The optical unit 5102 may further include a wavelength separation unit designed to separate light having a predetermined wavelength from the light from the biological sample S. The wavelength separation unit may be designed to selectively cause light having a predetermined wavelength or a predetermined wavelength range to reach the signal acquisition unit. The wavelength separation unit may include one or more of the following: a filter, a polarizing plate, a prism (Wollaston prism), and a diffraction grating that selectively pass light, for example. The optical component (s) included in the wavelength separation unit may be disposed in the optical path from the objective lens to the signal acquisition unit, for example. The wavelength separation unit is provided in the microscope device in a case where fluorescent observation is performed, or particularly, where an excitation light irradiation unit is included. The wavelength separation unit may be designed to separate fluorescence or white light from fluorescence.

### Signal Acquisition Unit

The signal acquisition unit 5103 may be designed to receive light from the biological sample S, and convert the light into an electrical signal, or particularly, into a digital electrical signal. The signal acquisition unit may be designed to be capable of acquiring data about the biological sample S, on the basis of the electrical signal. The signal acquisition unit may be designed to be capable of acquiring data of an image (a captured image, or particularly, a still image, a time-lapse image, or a moving image) of the biological sample S, or particularly, may be designed to acquire data of an image enlarged by the optical unit. The signal acquisition unit includes one or more image sensors, CMOSs, CCDs, or the like that include a plurality of pixels arranged in one- or two-dimensional manner. The signal acquisition unit may include an image sensor for acquiring a low-resolution image and an image sensor for acquiring a high-resolution image, or may include an image sensor for sensing for AF or the like and an image sensor for outputting an image for observation or the like. The image sensor may include not only the plurality of pixels, but also a signal processing unit (including one, two or three of the following: a CPU, a DSP, and a memory) that performs signal processing using pixel signals from the respective pixels, and an output control unit that controls outputting of image data generated from the pixel signals and processed data generated by the signal processing unit. Moreover, the imaging element can include an asynchronous event detection sensor that detects, as an event, that a luminance change of a pixel that photoelectrically converts incident light exceeds a predetermined threshold. The image sensor including the plurality of pixels, the signal processing unit, and the output control unit can be preferably designed as a one-chip semiconductor device.

### Control Unit

The control unit 5110 controls imaging being performed by the microscope device 5100. For the imaging control, the control unit can drive movement of the optical unit 5102 and/or the sample placement unit 5104, to adjust the positional relationship between the optical unit and the sample placement unit. The control unit 5110 can move the optical unit and/or the sample placement unit in a direction toward or away from each other (in the optical axis direction of the objective lens, for example). The control unit may also move the optical unit and/or the sample placement unit in any direction in a plane perpendicular to the optical axis direction. For the imaging control, the control unit may control the light irradiation unit 5101 and/or the signal acquisition unit 5103.

### Sample Placement Unit

The sample placement unit 5104 may be designed to be capable of securing the position of a biological sample on the sample placement unit, and may be a so-called stage. The sample placement unit 5104 may be designed to be capable of moving the position of the biological sample in the optical axis direction of the objective lens and/or in a direction perpendicular to the optical axis direction.

### Information Processing Unit

The information processing unit 5120 can acquire, from the microscope device 5100, data (imaging data or the like) acquired by the microscope device 5100. The information processing unit can perform image processing on the imaging data. The image processing may include color separation processing. The color separation processing may include a process of extracting data of the optical component of a predetermined wavelength or in a predetermined wavelength range from the imaging data to generate image data, or a process of removing data of the optical component of a predetermined wavelength or in a predetermined wavelength range from the imaging data. The image processing may also include an autofluorescence separation process for separating the autofluorescence component and the dye component of a tissue section, and a fluorescence separation process for separating wavelengths between dyes having different fluorescence wavelengths from each other. The autofluorescence separation process may include a process of removing the autofluorescence component from image information about another specimen, using an autofluorescence signal extracted from one specimen of the plurality of specimens having the same or similar properties.

The information processing unit 5120 may transmit data for the imaging control to the control unit 5110, and the control unit 5110 that has received the data may control the imaging being by the microscope device 5100 in accordance with the data.

The information processing unit 5120 may be designed as an information processing device such as a general-purpose computer, and may include a CPU, RAM, and ROM. The information processing unit may be included in the housing of the microscope device 5100, or may be located outside the housing. Further, the various processes or functions to be executed by the information processing unit may be realized by a server computer or a cloud connected via a network.

The method to be implemented by the microscope device 5100 to capture an image of the biological sample S may be appropriately selected by a person skilled in the art, in accordance with the type of the biological sample, the purpose of imaging, and the like. Examples of the imaging method are described below.

One example of the imaging method is as follows. The microscope device can first identify an imaging target region. The imaging target region may be identified so as to cover the entire region in which the biological sample exists, or may be identified so as to cover the target portion (the portion in which the target tissue section, the target cell, or the target lesion exists) of the biological sample. Next, the microscope device divides the imaging target region into a plurality of divided regions of a predetermined size, and the microscope device sequentially captures images of the respective divided regions. As a result, an image of each divided region is acquired.

As shown in FIG. 1, the microscope device identifies an imaging target region R that covers the entire biological sample S. The microscope device then divides the imaging target region R into 16 divided regions. The microscope device then captures an image of a divided region R1, and next captures one of the regions included in the imaging target region R, such as an image of a region adjacent to the divided region R1. After that, divided region imaging is performed until images of all the divided regions have been captured. Note that an image of a region other than the imaging target region R may also be captured on the basis of captured image information about the divided regions.

The positional relationship between the microscope device and the sample placement unit is adjusted so that an image of the next divided region is captured after one divided region is captured. The adjustment may be performed by moving the microscope device, moving the sample placement unit, or moving both. In this example, the imaging device that captures an image of each divided region may be a two-dimensional image sensor (an area sensor) or a one-dimensional image sensor (a line sensor). The signal acquisition unit may capture an image of each divided region via the optical unit. Further, images of the respective divided regions may be continuously captured while the microscope device and/or the sample placement unit is moved, or movement of the microscope device and/or the sample placement unit may be stopped every time an image of a divided region is captured. The imaging target region may be divided so that the respective divided regions partially overlap, or the imaging target region may be divided so that the respective divided regions do not overlap. A plurality of images of each divided region may be captured while the imaging conditions such as the focal length and/or the exposure time are changed.

Furthermore, the information processing device can combine a plurality of adjacent divided regions to generate image data of a wider region. As the combining process is performed on the entire imaging target region, an image of a wider region can be acquired with respect to the imaging target region. Furthermore, image data with lower resolution can be generated from the image of the divided region or the image subjected to the combining process.

Another example of the imaging method is as follows. The microscope device can first identify an imaging target region. The imaging target region may be identified so as to cover the entire region in which the biological sample exists, or may be identified so as to cover the target portion (the portion in which the target tissue section or the target cell exists) of the biological sample. Next, the microscope device scans a region (also referred to as a “divided scan region”) of the imaging target region in one direction (also referred to as a “scan direction”) in a plane perpendicular to the optical axis, and thus captures an image. After the scanning of the divided scan region is completed, the divided scan region next to the scan region is then scanned. These scanning operations are repeated until an image of the entire imaging target region is captured.

As shown in FIG. 3, the microscope device identifies a region (a gray portion) in which a tissue section of the biological sample S exists, as an imaging target region Sa. The microscope device then scans a divided scan region Rs of the imaging target region Sa in the Y-axis direction. After completing the scanning of the divided scan region Rs, the microscope device then scans the divided scan region that is the next in the X-axis direction. This operation is repeated until scanning of the entire imaging target region Sa is completed.

For the scanning of each divided scan region, the positional relationship between the microscope device and the sample placement unit is adjusted so that an image of the next divided scan region is captured after an image of one divided scan region is captured. The adjustment may be performed by moving the microscope device, moving the sample placement unit, or moving both. In this example, the imaging device that captures an image of each divided scan region may be a one-dimensional image sensor (a line sensor) or a two-dimensional image sensor (an area sensor). The signal acquisition unit may capture an image of each divided region via a magnifying optical system. Also, images of the respective divided scan regions may be continuously captured while the microscope device and/or the sample placement unit is moved. The imaging target region may be divided so that the respective divided scan regions partially overlap, or the imaging target region may be divided so that the respective divided scan regions do not overlap. A plurality of images of each divided scan region may be captured while the imaging conditions such as the focal length and/or the exposure time are changed.

Furthermore, the information processing device can combine a plurality of adjacent divided scan regions to generate image data of a wider region. As the combining process is performed on the entire imaging target region, an image of a wider region can be acquired with respect to the imaging target region. Furthermore, image data with lower resolution can be generated from the image of the divided scan region or the image obtained by the combining process.

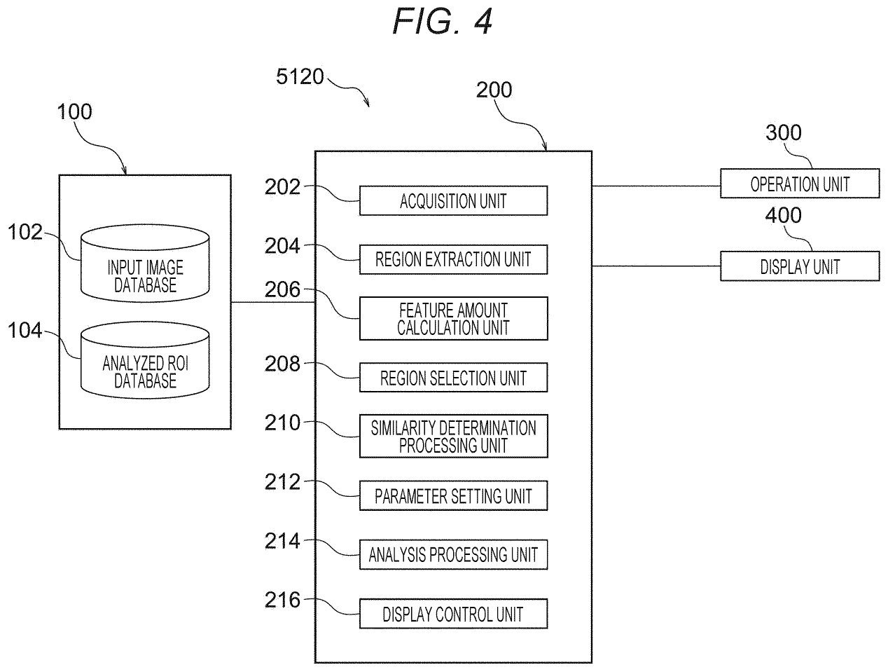

FIG. 4 is a block diagram illustrating a more detailed configuration example of an information processing unit 5120. As illustrated in FIG. 4, the information processing unit 5120 includes a storage unit 100, a processing unit 200, an operation unit 300, and a display unit 400. Note that the information processing unit 5120 according to the present embodiment corresponds to the information processing device.

As the storage unit 100, for example, a storage device such as a nonvolatile semiconductor memory or a hard disk drive is used. In the storage unit 100, various control parameters, programs, and the like according to the present embodiment are stored in advance. Furthermore, the storage unit 100 includes an input image database 102 and an analyzed ROI database 104.

The input image database 102 stores digital captured images captured by the microscope device 5100. The input image database 102 stores, for example, first region images captured at a plurality of different depths in the optical axis direction of the optical unit of the microscope device 5100. Moreover, the WSI image for each depth generated by performing stitching processing on a plurality of the first region images at the same depth is stored. Furthermore, it may be an image in a partial region indicated by annotation data (such as a tumor region indicated by a pathologist or a researcher) accompanying each image. The dyeability of these captured images may be hematoxylin & eosin (HE) staining, immunohistochemistry (IHC) staining, or fluorescent staining.

The analyzed ROI database 104 stores ROI region information associated with a captured image stored in the input image database 102, a feature amount calculated on the basis of an image in an ROI region, and a parameter associated with the feature amount in association with each other. Note that, in the analyzed ROI database 104, only the feature amount calculated on the basis of the image in the ROI region and the parameter associated with the feature amount may be stored. Furthermore, details of the feature amount and the like will be described later.

The processing unit 200 includes a central processing unit (CPU) and a microprocessor (MPU), and configures each processing unit by executing a program stored in the storage unit 100. The processing unit 200 analyzes a digital captured image captured by the microscope device 5100 and generates analysis information. Note that details of the processing unit 200 will be described later.

The program used by the processing unit 200 may be stored in the storage unit 100, or may be stored in a storage medium such as a digital versatile disc (DVD), a cloud computer, or the like. Furthermore, in the processing unit 200, the program may be executed by a central processing unit (CPU) or a microprocessor (MPU) using a random access memory (RAM) or the like as a work area, or may be executed by an integrated circuit such as an application specific integrated circuit (ASIC) or a field programmable gate array (FPGA).

The operation unit 300 includes, for example, a keyboard, a mouse, and the like. The operation unit 300 inputs an instruction signal corresponding to an operation of an observer (user), for example, a pathologist, to the processing unit 200.

The display unit 400 is, for example, a monitor. As will be described later, the display unit 400 displays a captured image, data related to analysis, a user interface (UI screen), and the like.

Here, details of the processing unit 200 will be described. The processing unit 200 includes an acquisition unit 202, a region extraction unit 204, a feature amount calculation unit 206, a region selection unit 208, a similarity determination processing unit 210, a parameter setting unit 212, an analysis processing unit 214, and a display control unit 216.

The acquisition unit 202 acquires a first image (target image) obtained by imaging a specimen tissue to be analyzed from the input image database 102. Alternatively, the acquisition unit 202 may directly acquire the target image from the microscope device 5100. Alternatively, the acquisition unit 202 may directly acquire the target image from another microscope device, a storage device, or the like via, for example, an in-hospital network.

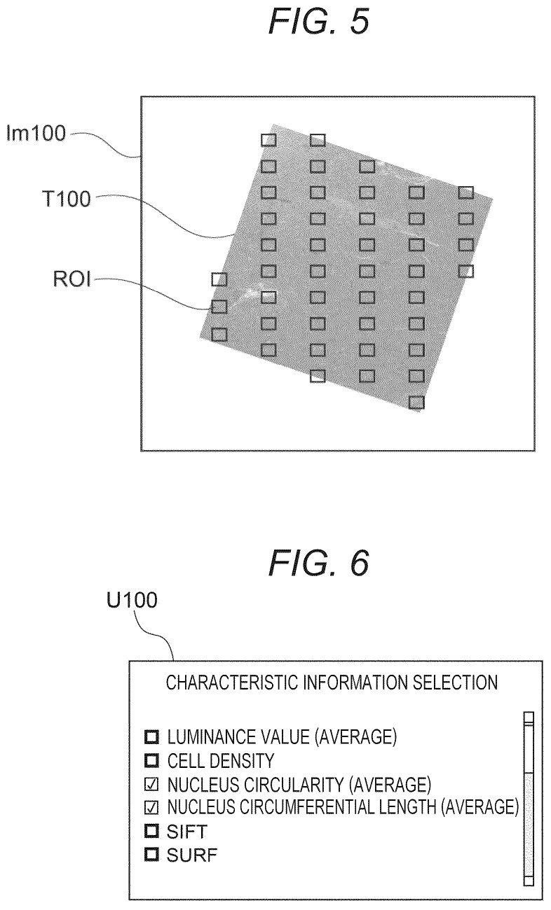

The region extraction unit 204 sets a region of interest (ROI) for calculating the feature amount in a captured image G100. FIG. 5 is a diagram illustrating an example of a captured image Im100 and a plurality of ROIs for calculating a feature amount. As illustrated in FIG. 5, the region extraction unit 204 extracts a sample region T100 from the captured image Im100, and sets a plurality of ROIs having a predetermined size in the sample region T100 at predetermined intervals. The size and interval of the ROI are set in the analyzed ROI database 104 in advance according to the feature amount to be calculated. Furthermore, the shape of the ROI is not limited, and may be, for example, a circular shape. Note that, in the present embodiment, the feature amount may be referred to as characteristic information. The characteristic information is obtained by quantifying at least one of various geometric characteristics including the tissue form present in the image and visual characteristics of various patterns including the tissue form, and the characteristic information is information including a feature amount.

The feature amount calculation unit 206 calculates a feature amount from each of the plurality of ROIs set by the region extraction unit 204. For example, the feature amount is a luminance value, cell density, nucleus circularity, a nucleus circumferential length, color information, a frequency characteristic, and Scale Invariant Feature Transform (SIFT), Speed Up Robust Features (SURF), local binary pattern (LBP), Accelerated KAZE (AKAZE), histogram of oriented gradients (HOG), and the like which are local feature amounts. In the present embodiment, these feature amounts are used, but the present embodiment is not limited thereto. For example, the feature amount may be a feature amount output from a neural network (NN) such as a convolutional neural network (CNN). As described above, the feature amount is only required to be a feature amount used for classification, identification, recognition, and the like of a pathological form by extracting image characteristics of a pathological image. For example, manually designed feature amounts include a color feature (luminance value, staining intensity, or the like), a shape feature (circularity, circumferential length, or the like), a density, a distance from a specific form, and a local feature amount (AKAZE, SIFT, HOG, SURF, LBP, or the like) of the specific form as described above. Furthermore, in a case where the data to be analyzed is IHC staining or fluorescent staining, information such as the number of positive/negative cells calculated in the tissue may be used.

FIG. 6 is a diagram illustrating an example of a user interface U100 for selecting characteristic information for selecting a feature amount. A pathologist or the like who is an observer selects a feature amount using the operation unit 300. In FIG. 6, an average value in the ROI of the nucleus circularity and an average value in the ROI of the nucleus circumferential length are selected. Hereinafter, in order to simplify the description, an example using an average value in the ROI of the nucleus circularity and an average value in the ROI of the nucleus circumferential length will be described, but the present embodiment is not limited thereto. For example, it is also possible to use a number of feature amounts on the order of several tens, several hundreds, or the like as the total feature amount. Note that the user interface U100 is displayed on the display unit 400 via the display control unit 216.

The region selection unit 208 selects a representative ROI on the basis of the distribution of the feature amount calculated from each of the pieces of image information in the plurality of ROIs set by the region extraction unit 204.

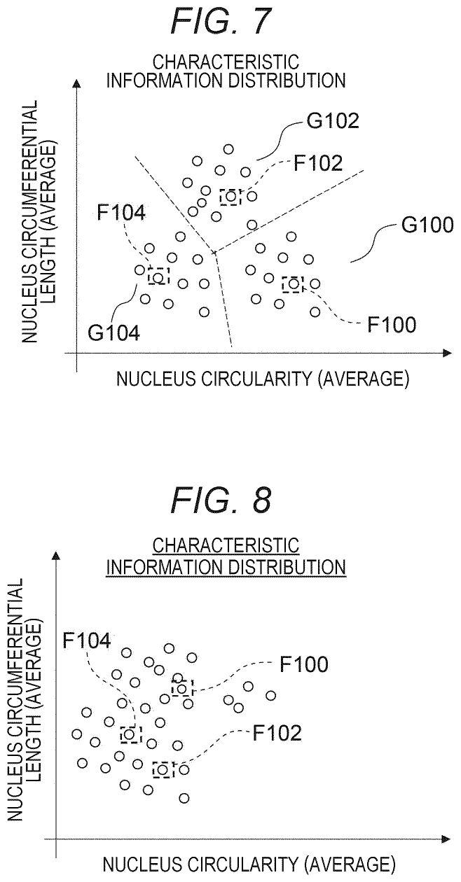

FIG. 7 is a diagram schematically illustrating a distribution of the feature amount of each ROI calculated by the feature amount calculation unit 206. The vertical axis represents the average value of the nucleus circumferential length for each ROI, and the horizontal axis represents the average value of the nucleus circularity for each ROI.

The region selection unit 208 clusters the feature amount and selects a representative ROI of each clustered region. As illustrated in FIG. 7, for example, the region selection unit 208 performs clustering by the K-Means method. The number of classifications can be set to any number. For example, the number of clusters may be manually designated by the user, or may be automatically determined by the region selection unit 208 by the elbow method, silhouette analysis, or the like.

For example, the region selection unit 208 selects a representative ROI from among clusters G100, G102, and G104. For example, the region selection unit 208 according to the present embodiment sets the ROI closest to the centroid of each of the clusters G100, G102, and G104 as the representative ROI, and selects F100, F102, and F104 as feature amounts of the representative ROI. As described above, the representative ROI and the feature amount corresponding to the representative ROI can be selected by the statistical processing. Thus, since the representative ROI and the feature amounts corresponding to the representative ROI are selected without depending on the experience of the observer, it is possible to improve the reproducibility in selectivity of the representative ROI and the feature amounts corresponding to the representative ROI.

The similarity determination processing unit 210 determines similarity between the feature amount of the ROI corresponding to the predetermined parameter stored in the analyzed ROI database 104 and the feature amount selected by the region selection unit 208. In a case of determining that the feature amount of the ROI corresponding to the predetermined parameter is similar to the feature amount selected by the region selection unit 208, the similarity determination processing unit 210 supplies the predetermined parameter to the parameter setting unit 212. Note that, in the present embodiment, determination of similarity between feature amounts may be referred to as comparison of feature amounts.

More specifically, the similarity determination processing unit 210 can perform the similarity determination on the basis of the feature amounts F100, F102, and F104 corresponding to the respective representative ROIs in the characteristic information space and a distance, a spatial vector, or the like between the feature amounts corresponding to the respective ROIs to be compared. For example, the similarity determination processing unit 210 determines that they are similar in a case where a Euclidean distance or a Mahalanobis distance, which is a geometric linear distance, is equal to or less than a threshold. Alternatively, for example, the similarity determination processing unit 210 may determine that they are similar in a case where a cosine similarity value indicating closeness in direction of the spatial vector is equal to or more than a threshold. Alternatively, after the similarity is compared in each characteristic information space as described above, similarity between sets may be used with a common term having close distance/vector. As an index at that time, a Jaccard coefficient, a Dice coefficient, or a Simpson coefficient, which are similarity indexes between sets, may be used.

FIG. 8 is a diagram schematically illustrating positions of the feature amounts F100, F102, and F104 of the representative ROI in a predetermined feature amount distribution. FIG. 8 illustrates an example in which the feature amounts F100, F102, and F104 are equal to or less than each feature amount in the feature amount distribution and a predetermined Mahalanobis distance. In such a case, the similarity determination processing unit 210 determines that the feature amounts F100, F102, and F104 are similar to the feature amount of the ROI corresponding to the parameter.

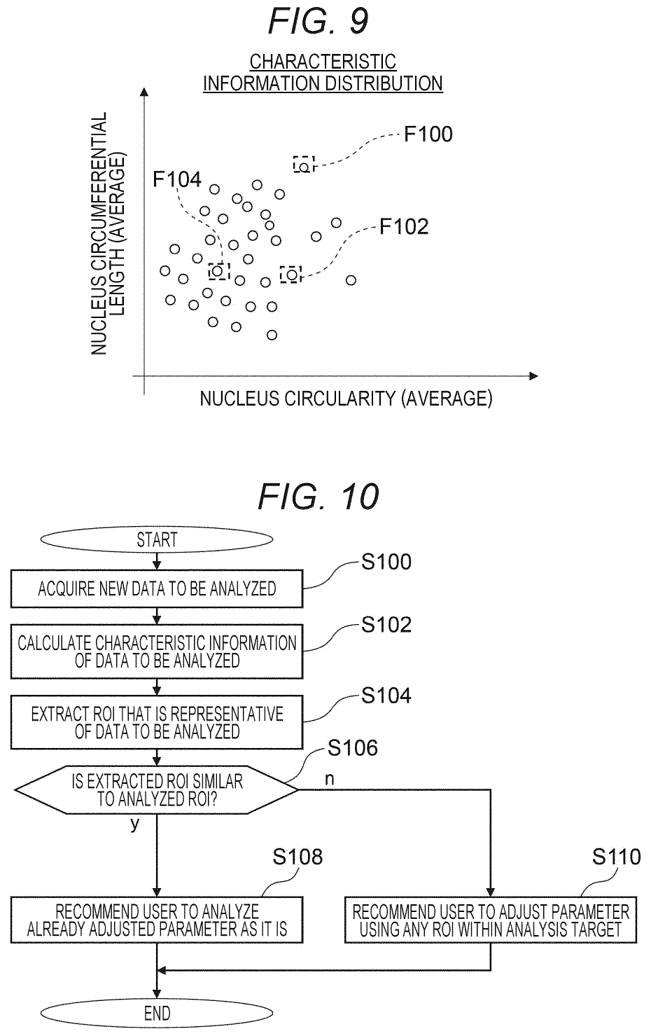

FIG. 9 is a diagram schematically illustrating a position in the feature amount distribution corresponding to another parameter of the feature amounts F100, F102, and F104 of the representative ROI. FIG. 9 illustrates an example in which the feature amount F104 is equal to or more than each feature amount in the feature amount distribution and a predetermined Mahalanobis distance. For example, in a case where even one of the representative feature amounts is equal to or more than a predetermined Mahalanobis distance with respect to each feature amount in the feature amount distribution, the similarity determination processing unit 210 determines that the feature amounts F100, F102, and F104 are not similar to the feature amount of the ROI corresponding to the parameter. In other words, the similarity determination processing unit 210 determines whether or not the pathological form in the image in the ROI corresponding to the parameter is similar to the pathological form in the image of the representative ROI. Note that the display control unit 216 may cause the display unit 400 to display the processing results of FIGS. 5 to 9 and the like. Thus, the observer can objectively grasp the processing result.

In a case of determining that the feature amounts F100, F102, and F104 are similar to the feature amount of the ROI corresponding to the parameter, the similarity determination processing unit 210 causes the display unit 400 via the display control unit to display a display form indicating to the user that analysis can be performed with the parameter being used. On the other hand, in a case of determining that the feature amounts F100, F102, and F104 are not similar to the feature amount of the ROI corresponding to the parameter, the similarity determination processing unit 210 causes the display unit 400 via the display control unit to display a display form prompting the observer (user) to readjust the parameter. Note that the similarity determination processing unit 210 according to the present embodiment corresponds to a determination processing unit.

The parameter setting unit 212 sets the parameter supplied from the similarity determination processing unit 210 in the analysis processing unit 214. Furthermore, the parameter setting unit 212 can also set a parameter that is set via the operation unit 300 in the analysis processing unit 214.

The analysis processing unit 214 analyzes the target image using the parameter set by the parameter setting unit 212. For example, the analysis processing unit 214 performs analysis processing according to each case using the set parameter. The analysis processing according to the present embodiment performs, for example, processing of extracting a lesion from a pathological image that is a target image, but is not limited thereto. For example, in the analysis processing, it is also possible to perform processing of extracting a target structure such as a cell nucleus from a pathological image that is a target image. Furthermore, for example, the analysis processing unit 214 may configure a plurality of image processing filters according to the set parameter, and perform analysis processing such as extracting a lesion by a combination of the plurality of image processing filters. Alternatively, processing including a combination of frequency processing and gradation conversion processing may be performed according to the set parameter.

The display control unit 216 performs processing of causing the display unit 400 to display various images. The display control unit 216 can also perform processing of reading a predetermined UI screen stored in the storage unit 100 and causing the display unit 400 to display the UI screen. In this case, an instruction signal input via the UI screen is supplied to each unit of the processing unit 200.

FIG. 10 is a flowchart illustrating a processing example of the information processing device 5120 according to a first embodiment. As illustrated in FIG. 10, the acquisition unit 202 acquires a target image to be processed from the input image database 102 (step S100).

Next, the region extraction unit 204 sets an ROI for calculating a feature amount in the captured image G100, the feature amount calculation unit 206 calculates a feature amount from each of the plurality of ROIs set by the region extraction unit 204, and the region selection unit 208 selects a representative ROI from each of the clusters G100, G102, and G104, for example (step S102).

Next, the similarity determination processing unit 210 determines similarity between the feature amount of the ROI corresponding to the predetermined parameter stored in the analyzed ROI database 104 and the feature amount selected by the region selection unit 208 (step S106). In a case of determining that they are similar (y in step S106), the similarity determination processing unit 210 causes the display unit 400 via the display control unit to display a display form indicating that analysis can be performed by the predetermined parameter adjusted in the past by the user (step S108).

On the other hand, in a case of determining that they are not similar (n in step S106), the similarity determination processing unit 210 causes the display unit 400 via the display control unit 216 to display a display form prompting the observer to readjust the parameter (step S110), and ends the entire processing.

As described above, the acquisition unit 202 acquires an image obtained by imaging a specimen tissue, and the analysis processing unit 214 analyzes the image using the predetermined parameter. In this case, the similarity determination processing unit 210 determines whether to allow the analysis processing to be performed with the predetermined parameter on the basis of the characteristic information of the tissue form in the image in the ROI existing in the image and the second characteristic information of the tissue form in the image corresponding to the predetermined parameter. Thus, it is possible to objectively determine whether or not the analysis processing can be performed on the image acquired by the acquisition unit 202 using the predetermined parameter. By extracting the ROI from the image and performing the similarity determination processing of the characteristic information based on the ROI in this manner, it is possible to objectively determine whether or not parameters adjusted in the past are applicable to a new image to be analyzed without experience or knowledge.

### Second Embodiment

The microscope system 5000 according to a second embodiment is different from the microscope system 5000 according to the first embodiment in that the processing unit 200 further includes a parameter adjustment control unit 218. Hereinafter, differences from the microscope system 5000 according to the first embodiment will be described.

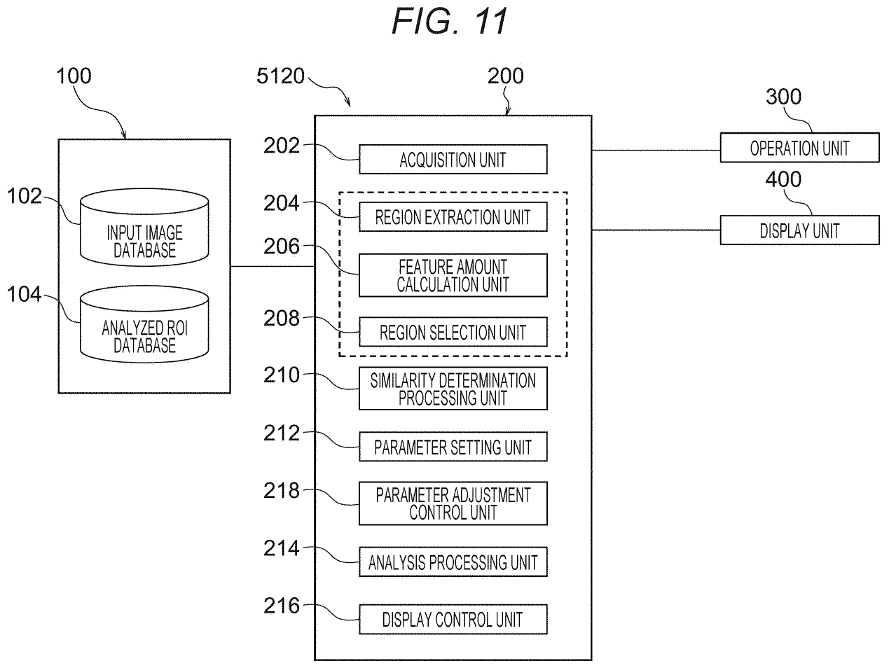

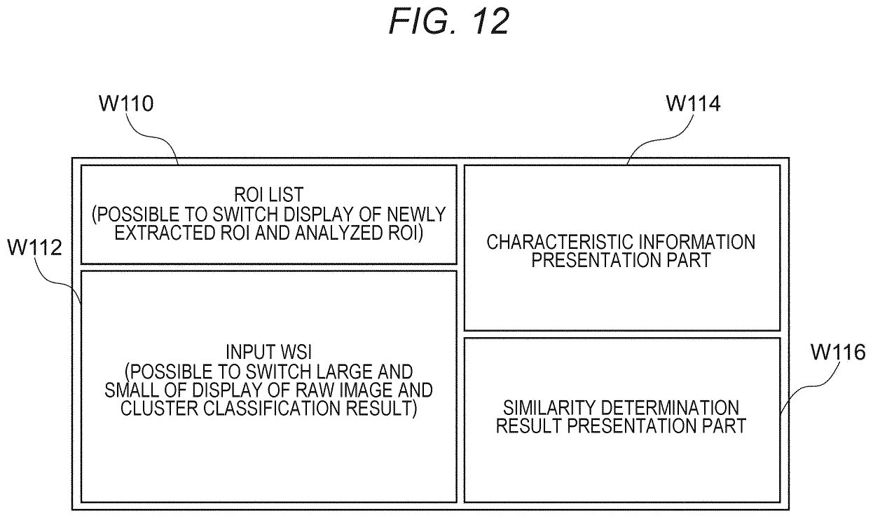

FIG. 11 is a block diagram illustrating a configuration example of the information processing unit 5120 according to the second embodiment. As illustrated in FIG. 11, the parameter adjustment control unit 218 is further included.

The parameter adjustment control unit 218 can perform control processing when performing parameter adjustment.

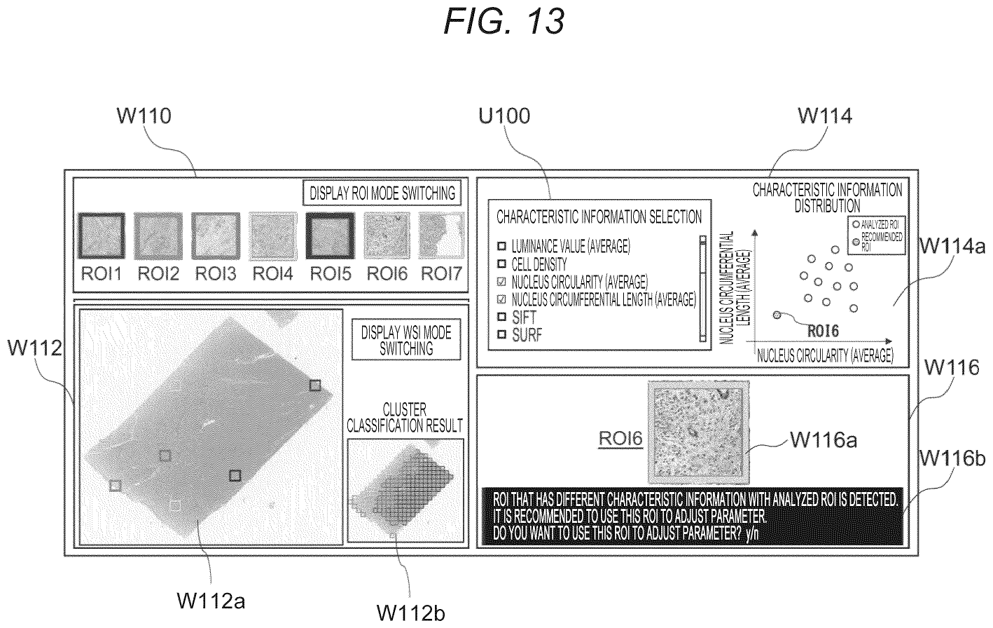

FIGS. 12 and 13 are diagrams illustrating screen examples to be displayed on the display unit 400 when the parameter adjustment control unit 218 performs the parameter adjustment control via the display control unit 216. FIG. 12 is a diagram illustrating an example of a screen region. As illustrated in FIG. 12, a screen region W110 is a region in which the image in the ROI selected by the region selection unit 208 is displayed, a screen region W112 is a region in which information regarding the WSI image is displayed, a screen region W114 is a region in which information regarding the characteristic information is displayed, and a screen region W116 is a region in which information regarding a similarity determination result is displayed.

FIG. 13 is a diagram illustrating an example in which specific information is displayed in each region illustrated in FIG. 12. As illustrated in FIG. 13, the parameter adjustment control unit 218 causes the display unit 400 to display the image in each of representative ROIs 1 to 7 selected by the region selection unit 208 in the screen region W110 via the display control unit 216. Thus, the observer can confirm the pathological form in the image in the representative ROI while comparing with a pathological form in an image in another representative ROI.

Furthermore, the parameter adjustment control unit 218 displays a pathological image W112a as the target image in the screen region W112 and illustrates the position of the representative ROI. Similarly, the parameter adjustment control unit 218 displays a result image W112b of the clustering by the region selection unit 208 on the screen region W112. In the result image W112b, regions close to the characteristic information are visualized by displaying the same color, or the like. Thus, the correspondence between the characteristic information and visual characteristics can be fed back to the user. By performing this feedback, for example, the user can monitor the validity of the cluster division number or the like.

Similarly, the parameter adjustment control unit 218 causes the display unit 400 via the display control unit 216 to display the UI screen U100 and a distribution image W114a of the feature amount of each ROI read from the storage unit 100 by the similarity determination processing unit 210 in the screen region W114. Furthermore, the ROI 6 determined not to be similar by the similarity determination processing unit 210 is also displayed in the distribution image W114a. Thus, the statistical positional relationship of the ROI 6 determined not to be similar can be confirmed on the feature amount distribution diagram. Therefore, the observer can objectively grasp the statistical position of the ROI 6 determined not to be similar.

Similarly, the parameter adjustment control unit 218 causes the display unit 400 via the display control unit 216 to display an enlarged view 116a of the image in the ROI 6 determined not to be similar to the screen region W116. Thus, the observer can confirm the pathological form of the ROI 6 determined not to be similar in more detail, and can grasp the cause of a distribution deviation of the feature amount on a statistical distribution.

Moreover, the parameter adjustment control unit 218 displays a display form W116b prompting the observer to readjust the parameter. Furthermore, the display form W116b is used to input a signal (y or n) indicating whether or not readjustment of the parameter is accepted.

With such a display form, the observer can objectively grasp the information of the ROI 6 determined not to be similar by the similarity determination processing unit 210. In this manner, by adding a mechanism for recommending an ROI dissimilar to the analyzed ROI, it is possible to select an ROI suitable for parameter adjustment without depending on the experience or pathological knowledge of the user.

Furthermore, in a case where the ROI 6 dissimilar to the analyzed ROI is detected by the similarity determination processing unit 210, the detected ROI 6 includes characteristic information that has not been analyzed before. That is, it is indicated that the recommended ROI 6 is more appropriate as an ROI for newly performing parameter adjustment. In this way, if it is an ROI whose characteristic information is separated from that of the analyzed ROI, it is considered that the usefulness of the parameter adjustment is high.

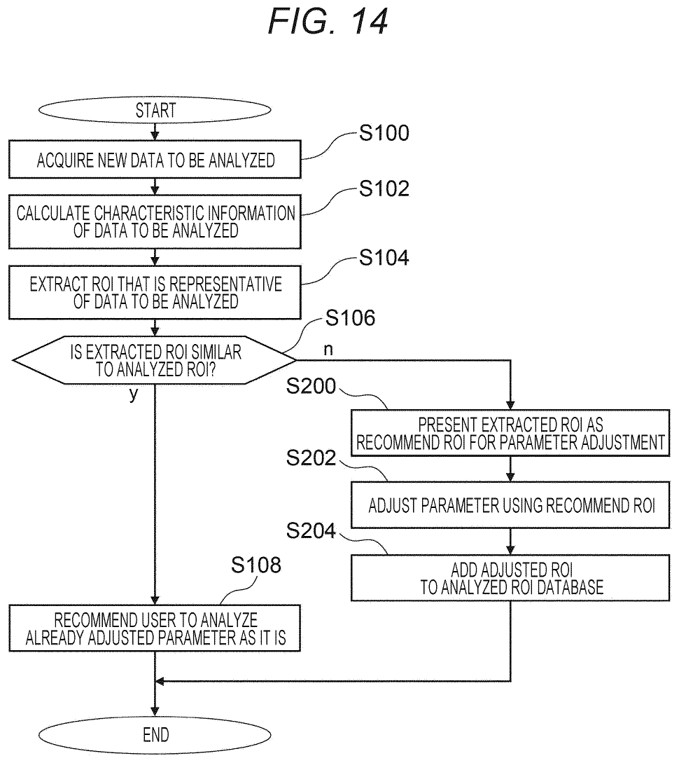

FIG. 14 is a flowchart illustrating a processing example of the information processing device 5120 according to the second embodiment. As illustrated in FIG. 14, in a case where the similarity determination processing unit 210 determines that they are not similar (n in step S106), the parameter adjustment control unit 218 causes the display unit 400 via the display control unit 216 to display the information of the ROI 6 recommended for readjustment of the parameter as, for example, a screen illustrated in FIG. 12 (step S200).

Next, the observer supplies a new parameter to the parameter setting unit 212 via the operation unit 300 (step S202). Then, the parameter adjustment control unit 218 stores the new parameter supplied via the operation unit 300 and the parameter setting unit 212 in association with the feature amount of the corresponding ROI 6 in the analyzed ROI database 104, and ends the entire processing.

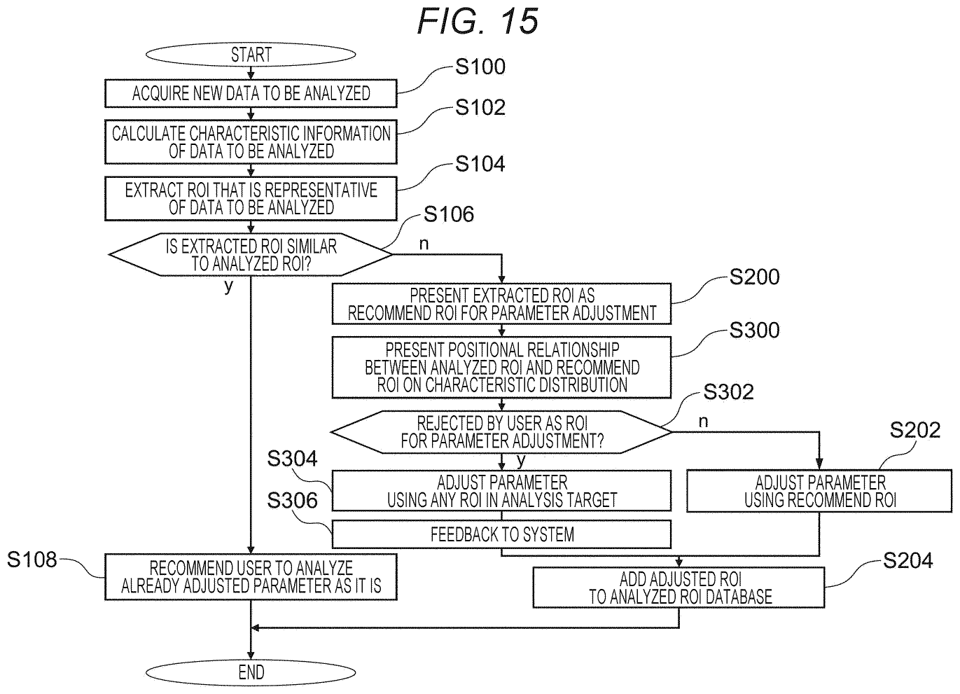

FIG. 15 is a flowchart illustrating a processing example of the information processing device 5120 in a case where the recommended ROI 6 is not employed (in a case where the recommended ROI 6 is not used for re-adjustment of parameters). As illustrated in FIG. 15, in a case where the similarity determination processing unit 210 determines that they are not similar (n in step S106), the parameter adjustment control unit 218 further causes the distribution image W114a of the feature amount of each ROI read from the storage unit 100 by the similarity determination processing unit 210 to be displayed (step S300). Then, the parameter adjustment control unit 218 determines whether or not a signal indicating that the recommended ROI 6 is not selected is input by the operation unit 300 via the display form W116b (see FIG. 13) (step S302).

In a case where a signal (n) indicating that the recommended ROI 6 is not selected is input (y in step S302), the parameter setting unit 212 sets a parameter related to any ROI in the analysis target in the analysis processing unit 214 and performs analysis processing.

Subsequently, the parameter adjustment control unit 218 feeds back imaging information related to the recommended ROI 6 to the microscope device 5100 (step S304). For example, an imaging condition for preventing acquisition of the feature amount related to the recommended ROI 6 is fed back to the microscope device 5100 (step S306). For example, the parameter adjustment control unit 218 supplies information for changing the wavelength band and illuminance in the light source of the microscope device 5100 to the microscope device 5100. Thus, for example, the microscope device 5100 can adjust the wavelength and illuminance of the light source in the light irradiation unit 5101 so as to suppress the acquisition of the statistical information related to the recommended ROI 6.

As described above, in the present embodiment, the parameter adjustment control unit 218 causes the display unit 400 via the display control unit 216 to display the recommended ROI dissimilar to the analyzed ROI. Thus, it is possible to select an ROI suitable for parameter adjustment without depending on the experience or pathological knowledge of the user.

### Third Embodiment

The microscope system 5000 according to a third embodiment is different from the microscope system 5000 according to the second embodiment in that the processing unit 200 further includes a determination reference learning unit 220. Hereinafter, differences from the microscope system 5000 according to the second embodiment will be described.

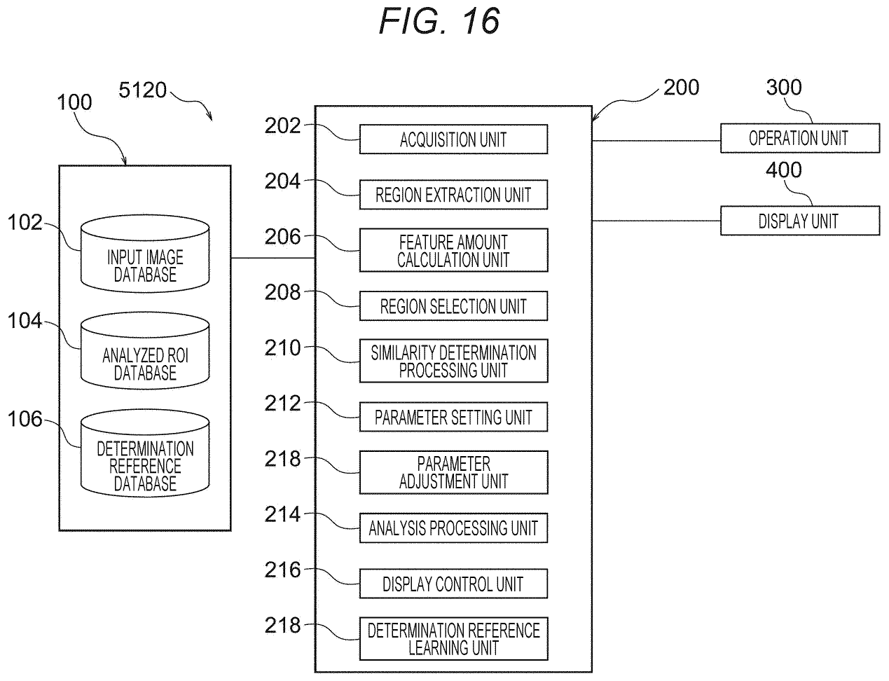

FIG. 16 is a block diagram illustrating the configuration example of the information processing device 5120 according to the third embodiment. As illustrated in FIG. 16, the determination reference learning unit 220 is further included.

As described above, in a case where the representative ROI 6 (see FIG. 13) is at a position deviated in the existing feature amount distribution, that is, in a case where the feature amount based on the representative ROI 6 and each feature amount are not within the predetermined distance, the similarity determination processing unit 210 prompts for the adjustment of the parameter. On the other hand, the representative ROI 6 may not be employed for the adjustment of the parameter. In such a case, if the position of the feature amount of the representative ROI 6 in the feature amount space is not brought close to the existing feature amount distribution, there is a high possibility that the feature amount similar to the representative ROI 6 is employed for the adjustment of the parameter. Accordingly, the determination reference learning unit 220 performs processing of relearning the similarity determination reference so that the feature amount of the representative ROI 6 approaches the existing feature amount distribution on the feature amount space.

The determination reference learning unit 220 causes, for example, the similarity determination reference of the similarity determination processing unit 210 to be relearned. As a relearning method, the determination reference learning unit 220 automatically updates the thresholds related to the Mahalanobis distance and the spatial vector used by the similarity determination processing unit 210. Thus, the threshold can be updated so that the statistic of the representative ROI 6 that has been rejected is determined to be “similar” to the analyzed ROI. Moreover, the learning may be performed so that the feature amount of the representative ROI 6 that has been rejected is plotted at a position close to the analyzed ROI in the distance space where the feature amount of the representative ROI 6 is generated by using deep metric learning or the like. In this way, it is possible to incorporate pathological knowledge or the like possessed by the user into the similarity determination and control the similarity determination reference of the system.

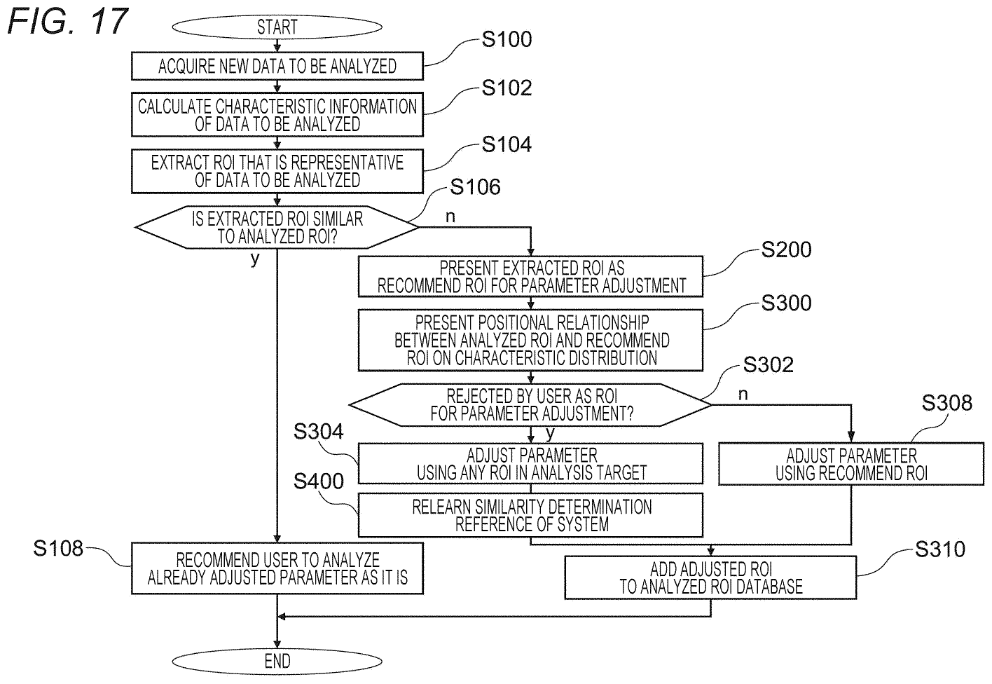

FIG. 17 is a flowchart illustrating a relearning example of the information processing device 5120 in a case where the recommended ROI 6 is not employed. As illustrated in FIG. 17, in a case where the signal indicating that the recommended ROI 6 is not selected is input (y in step S302), the determination reference learning unit 220 causes, for example, the similarity determination reference of the similarity determination processing unit 210 to be relearned (step S400).

As described above, according to the present embodiment, in a case where the similarity determination processing unit 210 determines that the representative ROI 6 (see FIG. 13) is located at a deviated position in the existing feature amount distribution, and the user does not employ the representative ROI 6 for the adjustment of the parameter, the determination reference learning unit 220 performs processing of relearning the similarity determination reference so that the feature amount of the representative ROI 6 approaches the existing feature amount distribution on the feature amount space. This suppresses the similarity determination processing unit 210 from selecting an ROI having a feature amount similar to that of the representative ROI 6 as the recommended ROI. As described above, in addition to the characteristic information, the pathological knowledge or the like possessed by the user can be incorporated into the similarity determination, and the similarity determination reference of the system can also be controlled.

### Fourth Embodiment

The microscope system 5000 according to a fourth embodiment is different from the microscope system 5000 according to the second embodiment in that the similarity determination processing unit 210 determines whether there is similar information in the characteristic information stored in the storage unit 100 in a case of determining that the representative ROI 6 (see FIG. 13) is at a deviated position in the existing feature amount distribution. Hereinafter, differences from the microscope system 5000 according to the second embodiment will be described.

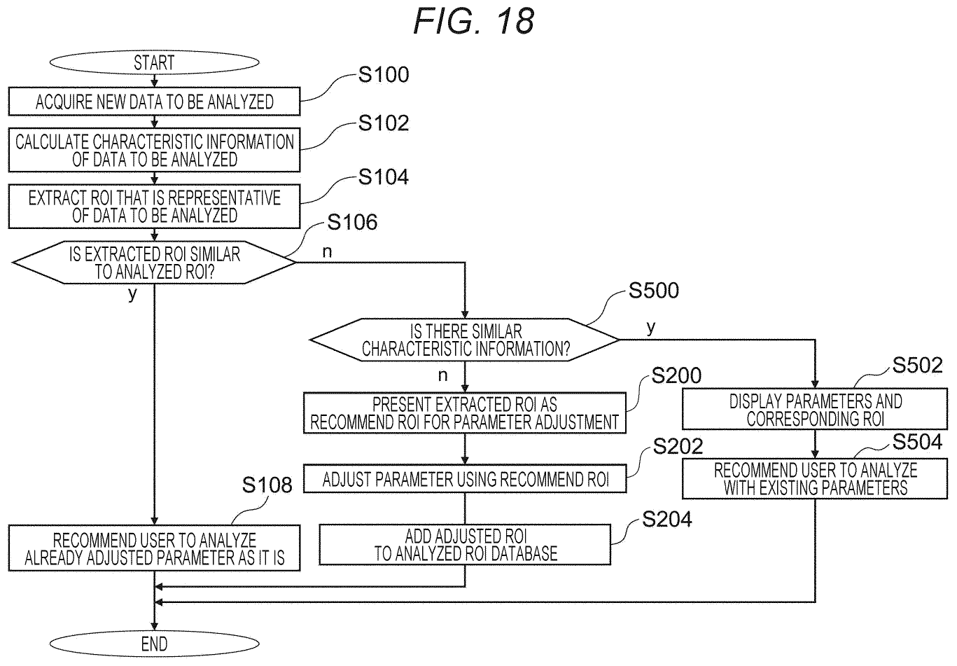

FIG. 18 is a flowchart illustrating an example of determining whether there is similar information in the characteristic information stored in the storage unit 100. As illustrated in FIG. 18, in a case where the similarity determination processing unit 210 determines that there is no similarity (n in step S106), the parameter adjustment control unit 218 determines whether or not there is a feature amount corresponding to the recommended ROI 6 in the storage unit 100 (step S500). In a case where the parameter adjustment control unit 218 determines that there is a similar feature amount (y in step S500), the parameter adjustment control unit 218 causes the display unit 400 via the display control unit 216 to display the parameter associated with the similar feature amount and the image in the ROI associated with the parameter (step S502). Then, a display form in which analysis with the displayed parameters is recommended to the user is displayed.

On the other hand, in a case where it is not determined (n in step S500), steps S200 to S204 described above are executed.

As described above, according to the present embodiment, in a case where the similarity determination processing unit 210 determines that the representative ROI 6 (see FIG. 13) is not similar in the existing feature amount distribution, it is determined whether or not the feature amount corresponding to the recommended ROI 6 exists in the storage unit 100. Thus, in a case where there is the feature amount corresponding to the recommended ROI 6 in the storage unit 100, it is possible to use the parameter associated with the corresponding feature amount, and it is possible to further improve the processing efficiency.

### Modification of Fourth Embodiment

The microscope system 5000 according to the fifth embodiment is different from the microscope system 5000 according to the fourth embodiment in that the determination distance used in the similarity determination processing unit 210 is changed according to the number of pieces of characteristic information stored in the storage unit 100. Hereinafter, differences from the microscope system 5000 according to the fourth embodiment will be described.

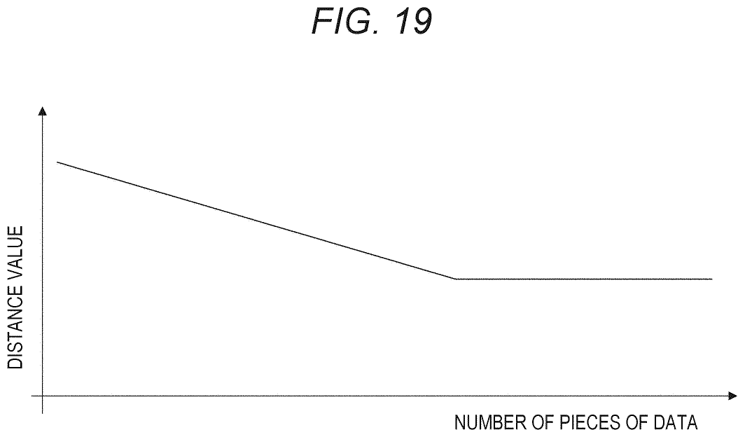

FIG. 19 is a diagram illustrating an example of the determination distance used by the similarity determination processing unit 210. The horizontal axis represents the number of pieces of characteristic information stored in the storage unit 100, and the vertical axis represents the determination distance used by the similarity determination processing unit 210. As illustrated in FIG. 19, the similarity determination processing unit 210 shortens the predetermined Mahalanobis distance used for determination as the characteristic information associated with the parameter increases.

The number of pieces of characteristic information associated with the new parameter increases as the number of captured images increases. Therefore, as the characteristic information increases, by shortening the predetermined Mahalanobis distance used for determination, it is possible to make the expansion of the determination region of the similarity of the representative ROI constant. Thus, the determination processing accuracy of the similarity determination processing unit 210 can be suppressed with respect to variation in the number of pieces of data of the characteristic information.

Note that the present technology can have the following configurations.

(1) An information processing program, including:
an acquisition step of acquiring a first image obtained by imaging a specimen tissue;
an analysis processing step of performing analysis processing on the first image using a predetermined parameter; and
a determination step of determining whether to allow the analysis processing to be performed with the predetermined parameter on the basis of first characteristic information of a tissue form present in the first image and second characteristic information of a tissue form corresponding to the predetermined parameter.

(2) The information processing program according to (1), in which the determination step determines not to allow the analysis processing to be performed with the predetermined parameter in a case where the first characteristic information and the second characteristic information are not within a predetermined distance on a statistical distribution.

(3) The information processing program according to (2), further including a display control step of causing a display unit to display a display form in which setting of a new parameter for the analysis processing step is recommended in a case where the determination step determines not to allow the analysis processing to be performed with the predetermined parameter.

(4) The information processing program according to (3), in which
the first characteristic information is generated on the basis of an image in a predetermined region of the first image, and
at least one of an image in the predetermined region or an image indicating the predetermined region is displayed as the display form.

(5) The information processing program according to (3), in which a language related to recommending setting of a parameter is displayed as the display form.

(6) The information processing program according to any one of (3) to (5), in which the display control step causes the display unit to display an image indicating position information of the first characteristic information in the statistical distribution and the display form side by side.

(7) The information processing program according to any one of (3) to (6), in which the first characteristic information is a plurality of pieces of characteristic information selected from a plurality of pieces of characteristic information based on a plurality of processing regions in the first image, and
the display control step causes the display unit to display images in a processing region corresponding to the selected plurality of pieces of characteristic information side by side.

(8) The information processing program according to any one of (3) to (6), in which
the first characteristic information is a plurality of pieces of characteristic information selected from each of clustered regions by clustering a plurality of pieces of characteristic information based on each of a plurality of processing regions in the first image into a plurality of regions, and
the display control step displays the processing regions of each of the clustered regions on the first image in association with each other.

(9) The information processing program according to any one of (1) to (6), in which
the first characteristic information is a plurality of pieces of characteristic information selected from a plurality of pieces of characteristic information based on a plurality of processing regions in the first image, and
the determination step changes a determination reference in such a manner that the first characteristic information is not selected in a case where it is determined not to allow the analysis processing to be performed with the predetermined parameter and a parameter based on a processing region corresponding to the first characteristic information is not used for the analysis processing.

(10) The information processing program according to any one of (1) to (6), in which
the first characteristic information is a plurality of pieces of characteristic information selected from a plurality of pieces of characteristic information based on a plurality of processing regions in the first image, and
the determination step changes an imaging condition of an imaging device that has captured the first image in such a manner that the first characteristic information is not selected in a case where it is determined not to allow the analysis processing to be performed with the predetermined parameter and a parameter based on a processing region corresponding to the first characteristic information is not used for the analysis processing.

(11)

The information processing program according to any one of (2) to (10), in which the second characteristic information is a plurality of pieces of characteristic information calculated from different captured images.

(12)

The information processing program according to (9), in which the determination step changes the predetermined distance according to a number of pieces of the second characteristic information corresponding to the predetermined parameter.

(13) The information processing program according to any one of (1) to (12), further including
a storage step of storing a plurality of parameters used in the analysis processing step in a storage unit in association with the second characteristic information corresponding to the parameters, in which
in a case where the determination step determines not to allow the analysis processing to be performed with the predetermined parameter, the determination step causes the storage unit to store a new parameter of the analysis processing step corresponding to the first characteristic information in association with the first characteristic information.

(14) The information processing program according to (13), in which the determination step determines whether or not characteristic information similar to the first characteristic information is stored in the storage unit in a case where it is determined not to allow the analysis processing to be performed with the predetermined parameter, and in a case where the characteristic information is not stored, the determination step causes the storage unit to store a new parameter of the analysis processing step corresponding to the first characteristic information.

(15) The information processing program according to (14), in which in a case where it is determined that the characteristic information similar to the first characteristic information is stored in the storage unit, the determination step allows the analysis processing to be performed on the basis of a parameter corresponding to the similar characteristic information.

(16) The information processing program according to (1), in which the determination step allows the analysis processing to be performed with the predetermined parameter in a case where the first characteristic information and the second characteristic information are within a predetermined distance on a statistical distribution.

(17) The information processing program according to (1), further including a display control step of causing a display unit to display a display form indicating that it is possible to perform the analysis processing with the predetermined parameter in a case where the determination step determines to allow the analysis processing to be performed with the predetermined parameter.

(18) The information processing program according to (2), in which the first characteristic information and the second characteristic information are at least one of a luminance value, cell density, cell circularity, a cell circumferential length, or a local feature amount, and the distance is at least one of a Mahalanobis distance or a Euclidean distance.

(19) An information processing device, including:
an acquisition unit that acquires a first image obtained by imaging a specimen tissue;
an analysis processing unit that performs analysis processing on the first image using a predetermined parameter; and
a determination unit that determines whether to allow the analysis processing to be performed with the predetermined parameter on the basis of first characteristic information of a tissue form present in the first image and second characteristic information of a tissue form corresponding to the predetermined parameter.

(20) An information processing method, including:
an acquisition step of acquiring a first image obtained by imaging a specimen tissue;
an analysis processing step of performing analysis processing on the first image using a predetermined parameter; and
a determination step of determining whether to allow the analysis processing to be performed with the predetermined parameter on the basis of first characteristic information of a tissue form present in the first image and second characteristic information of a tissue form corresponding to the predetermined parameter.

(21) A microscope system, including:
a microscope device that acquires a first image obtained by imaging a specimen tissue; and
an information processing device, in which
the information processing device includes
an acquisition unit that acquires a first image obtained by imaging a specimen tissue,
an analysis processing unit that performs analysis processing on the first image using a predetermined parameter, and
a determination unit that determines whether to allow the analysis processing to be performed with the predetermined parameter on the basis of first characteristic information of a tissue form present in the first image and second characteristic information of a tissue form corresponding to the predetermined parameter.

Aspects of the present disclosure are not limited to the above-described individual embodiments, but include various modifications that can be conceived by those skilled in the art, and the effects of the present disclosure are not limited to the above-described contents. That is, various additions, modifications, and partial deletions are possible without departing from the conceptual idea and spirit of the present disclosure derived from the matters defined in the claims and equivalents thereof.

### REFERENCE SIGNS LIST

100 Storage unit

200 Processing unit

202 Acquisition unit

204 Region extraction unit

206 Feature amount calculation unit

208 Region selection unit

210 Similarity determination processing unit

212 Parameter setting unit

214 Analysis processing unit

216 Display control unit

5000 Microscope system

400 Display unit

5120 Information processing device

5100 Microscope device

1. An information processing program for causing a computer to execute:
an acquisition step of acquiring a first image obtained by imaging a specimen tissue;
an analysis processing step of performing analysis processing on the first image using a predetermined parameter; and
a determination step of determining whether to allow the analysis processing to be performed with the predetermined parameter on a basis of first characteristic information of a tissue form present in the first image and second characteristic information of a tissue form corresponding to the predetermined parameter.

2. The information processing program according to claim 1, wherein the determination step determines not to allow the analysis processing to be performed with the predetermined parameter in a case where the first characteristic information and the second characteristic information are not within a predetermined distance on a statistical distribution.

3. The information processing program according to claim 2, further comprising a display control step of causing a display unit to display a display form in which setting of a new parameter for the analysis processing step is recommended in a case where the determination step determines not to allow the analysis processing to be performed with the predetermined parameter.

4. The information processing program according to claim 3, wherein
the first characteristic information is generated on a basis of an image in a predetermined region of the first image, and
at least one of an image in the predetermined region or an image indicating the predetermined region is displayed as the display form.

5. The information processing program according to claim 3, wherein a language related to recommending setting of a parameter is displayed as the display form.

6. The information processing program according to claim 3, wherein the display control step causes the display unit to display an image indicating position information of the first characteristic information in the statistical distribution and the display form side by side.

7. The information processing program according to claim 3, wherein
the first characteristic information is a plurality of pieces of characteristic information selected from a plurality of pieces of characteristic information based on a plurality of processing regions in the first image, and
the display control step causes the display unit to display images in a processing region corresponding to the selected plurality of pieces of characteristic information side by side.

8. The information processing program according to claim 3, wherein
the first characteristic information is a plurality of pieces of characteristic information selected from each of clustered regions by clustering a plurality of pieces of characteristic information based on each of a plurality of processing regions in the first image into a plurality of regions, and
the display control step displays the processing regions of each of the clustered regions on the first image in association with each other.

9. The information processing program according to claim 1, wherein
the first characteristic information is a plurality of pieces of characteristic information selected from a plurality of pieces of characteristic information based on a plurality of processing regions in the first image, and
the determination step changes a determination reference in such a manner that the first characteristic information is not selected in a case where it is determined not to allow the analysis processing to be performed with the predetermined parameter and a parameter based on a processing region corresponding to the first characteristic information is not used for the analysis processing.

10. The information processing program according to claim 1, wherein
the first characteristic information is a plurality of pieces of characteristic information selected from a plurality of pieces of characteristic information based on a plurality of processing regions in the first image, and
the determination step changes an imaging condition of an imaging device that has captured the first image in such a manner that the first characteristic information is not selected in a case where it is determined not to allow the analysis processing to be performed with the predetermined parameter and a parameter based on a processing region corresponding to the first characteristic information is not used for the analysis processing.

11. The information processing program according to claim 2, wherein the second characteristic information is a plurality of pieces of characteristic information calculated from different captured images.

12. The information processing program according to claim 9, wherein the determination step changes the predetermined distance according to a number of pieces of the second characteristic information corresponding to the predetermined parameter.

13. The information processing program according to claim 1, further comprising
a storage step of storing a plurality of parameters used in the analysis processing step in a storage unit in association with the second characteristic information corresponding to the parameters, wherein
in a case where the determination step determines not to allow the analysis processing to be performed with the predetermined parameter, the determination step causes the storage unit to store a new parameter of the analysis processing step corresponding to the first characteristic information in association with the first characteristic information.

14. The information processing program according to claim 13, wherein the determination step determines whether or not characteristic information similar to the first characteristic information is stored in the storage unit in a case where it is determined not to allow the analysis processing to be performed with the predetermined parameter, and in a case where the characteristic information is not stored, the determination step causes the storage unit to store a new parameter of the analysis processing step corresponding to the first characteristic information.

15. The information processing program according to claim 14, wherein in a case where it is determined that the characteristic information similar to the first characteristic information is stored in the storage unit, the determination step allows the analysis processing to be performed on a basis of a parameter corresponding to the similar characteristic information.

16. The information processing program according to claim 1, wherein the determination step allows the analysis processing to be performed with the predetermined parameter in a case where the first characteristic information and the second characteristic information are within a predetermined distance on a statistical distribution.

17. The information processing program according to claim 1, further comprising a display control step of causing a display unit to display a display form indicating that it is possible to perform the analysis processing with the predetermined parameter in a case where the determination step determines to allow the analysis processing to be performed with the predetermined parameter.

18. The information processing program according to claim 2, wherein the first characteristic information and the second characteristic information are at least one of a luminance value, cell density, nucleus circularity, a nucleus circumferential length, color information, a frequency characteristic, or a local feature amount, or the distance is at least one of a Mahalanobis distance or a Euclidean distance.

19. An information processing device, comprising:
an acquisition unit that acquires a first image obtained by imaging a specimen tissue;
an analysis processing unit that performs analysis processing on the first image using a predetermined parameter; and
a determination unit that determines whether to allow the analysis processing to be performed with the predetermined parameter on a basis of first characteristic information of a tissue form present in the first image and second characteristic information of a tissue form corresponding to the predetermined parameter.

20. An information processing method, comprising:
an acquisition step of acquiring a first image obtained by imaging a specimen tissue;
an analysis processing step of performing analysis processing on the first image using a predetermined parameter; and
a determination step of determining whether to allow the analysis processing to be performed with the predetermined parameter on a basis of first characteristic information of a tissue form present in the first image and second characteristic information of a tissue form corresponding to the predetermined parameter.

21. A microscope system, comprising:
a microscope device that acquires a first image obtained by imaging a specimen tissue; and
an information processing device, wherein
the information processing device includes
an acquisition unit that acquires a first image obtained by imaging a specimen tissue,
an analysis processing unit that performs analysis processing on the first image using a predetermined parameter, and
a determination unit that determines whether to allow the analysis processing to be performed with the predetermined parameter on a basis of first characteristic information of a tissue form present in the first image and second characteristic information of a tissue form corresponding to the predetermined parameter.
